(19) United States
(12) Patent Application Publication
(10) Pub. No.: US 2007/0134795 A1
(43) Pub. Date: Jun. 14, 2007

(54) Flp-mediated recombination

(76) Inventors: Holly Prentice, Carlisle, MA (US); Louisa Caamano, Medford, MA (US)

Correspondence Address: FISH & RICHARDSON PC, P.O. BOX 1022, MINNEAPOLIS, MN, 55440-1022, US

(21) Appl. No.: 10/575,696

(22) PCT Filed: Oct. 14, 2004
(86) PCT No.: PCT/US04/33868

§ 371 Date: Dec. 4, 2006

Related U.S. Application Data
(60) Provisional application No. 60/511,610, filed on Oct. 14, 2003.

(51) Int. Cl. C12N 5/06

(52) U.S. Cl. 435/358; 435/455

(57) ABSTRACT

Compositions and methods useful for homologous recombination and stable integration of a polynucleotide into a host cell are provided. The disclosed compositions and methods provide a rapid and efficient method for stably integrating an exogenous polynucleotide into a host cell and selecting such transformed cells.

### TECHNICAL FIELD

This invention relates to recombination vectors and recombination cassettes, and more particularly to methods, compositions, and systems for expression of an exogenous molecule in an organism or host cell.

### BACKGROUND

The introduction of nucleic acid molecules, polypeptides, and peptides into target cells and tissues is being used both as a therapeutic delivery system as well as in the production of therapeutic molecules in vitro. The applicability of this approach has increased with the further understanding of host cells and the molecular biology of cell division, differentiation, and expression mechanisms.

Gene targeting by means of homologous recombination between homologous exogenous DNA and endogenous chromosomal sequences has proven to be an extremely valuable way to create deletions, or insertions, design mutations, correct gene mutations, introduce transgenes, or make other genetic modifications.

### SUMMARY

The invention provides a recombination cassette comprising a promoter/enhancer region; a polynucleotide of interest; a polyA signal domain; an FRT recombination domain; and a dhfr polynucleotide, wherein the promoter/enhancer region, the polynucleotide of interest and the polyA signal domain are operably linked.

The invention also provide a recombination vector comprising a recombination cassette comprising a promoter/enhancer region; a polynucleotide of interest; a polyA signal domain; an FRT recombination domain; and a dhfr polynucleotide, wherein the promoter/enhancer region, the polynucleotide of interest and the polyA signal domain are operably linked. In one embodiment, the vector comprises a sequence as set forth in SEQ ID NO:1 or 2. In yet another embodiment, the recombination vector, further comprises a second promoter/enhancer region; a second polynucleotide of interest; and a second polyA signal domain, wherein the second promoter/enhancer region, the second polynucleotide of interest, and the second polyA signal are operably linked.

The invention fierier provides a host cell containing one or more copies of a stably integrated recombination cassette of the invention. In one embodiment, the host cell is adapted for growth in suspension and/or in serum-free medium.

The invention also provides a recombination system. The recombination system comprises an expression plasmid containing one or more FRT recombination domains; and a host cell containing one or more FRT sites. In one embodiment, the host cell is a CHO cell including, for example, a CHO-DG44 cell. In some embodiment, the host cell is adapted for growth in suspension and/or in serum-free medium.

The invention further provides a kit comprising a vector and/or recombination cassette of the invention and a host cell comprising an FRT site.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 shows a FLP Recombination Target (FRT) site and the process of introduction into the genome of CHO host cells. An expression vector containing a polynucleotide of interest can be transfected (stable integrated) into the genome via FLP recombinase-mediated DNA recombination at the FRT site.

FIG. 2 shows a map of a recombination vector of the invention (corresponding to SEQ ID NO:1).

FIG. 3 shows a map of a recombination vector of the invention (corresponding to SEQ ID NO:2) comprising a vector that has two insertion sites (i.e., the multiple cloning sites beginning at 1309 and at 6370).

FIG. 4 shows a plasmid map of pFRTlacZeo, which was used to generate the CHO-DG44 Flp-In cell line.

FIG. 5 shows a plasmid map of pFRTlacZeo showing the probe used to identify the presence of FRT in transfected host cells.

FIG. 6 shows a representative photo of 17 of the >100 examined cell lines screened by Southern blot. Each lane contains 10 μg of genomic DNA from a transfected cell line. As seen by the different size bands, it is evident that many of these candidate host cell lines have had the FRT sequence integrated into the CHO-DG44 at different sites. Furthermore, an example of multiple integration sites of the FRT, as seen by multiple bands, is visible in lane 11.

FIG. 7 shows potential candidates that were tested again to confirm a single copy of FRT cassette. Lane 1 contains one μg pFRT/lacZeo, which serves as a positive control. Results of 9 of the 35 candidate cell lines are shown. Each lane contains 10 μg of genomic DNA from a transfected cell line. Only single bands are seen, confirming single integration. Furthermore, the various sized bands suggest differing integration locations of the FRT sites. The hybridization bands are very weak as a consequence of only a single copy of the FRT sequence being present in the CHO-DG44 genome.

FIGS. 8A and 8B show the nucleotide sequence of SEQ ID NO:1.

FIGS. 9A and 9B shows the nucleotide sequence of SEQ ID NO:2.

### DETAILED DESCRIPTION

The invention provides recombination cassettes and vectors that are useful for homologous recombination and stable integration of a desired polynucleotide (i.e., a polynucleotide of interest) into a host cell. The invention provides a polynucleotide construct that comprises sequences homologous to an endogenous chromosomal polynucleotide adequate to permit homologous recombination to a site in the chromosomal DNA of a host cell, a polynucleotide encoding a selectable marker and a cloning site, and may further comprise regulatory elements. The methods, systems and compositions of the invention provide a powerful tool for generating quantities of proteins with minimal work to generate a stable cell line.

In one aspect of the invention, there is provided a polynucleotide comprising a recombination cassette that includes a cytomegalovirus (CMV) transcriptional regulatory region, a variable length intervening sequence (e.g., from intron A of CMV), a polynucleotide of interest, a recombination domain, and a polyadenylation signal domain. The invention further relates to processes and expression vectors for producing and recovering heterologous polypeptides from host cells.

In another aspect, a recombination cassette of the invention includes, operably linked, (i) a CMV major immediate early 1 (IE1) promoter/enhancer region and a variable length intervening sequence (e.g., derivative of intron A), (ii) a polynucleotide of interest, (iii) a first polyadenylation signal domain (e.g., BHG or hGH polyA), (iv) a recombination domain (e.g., an FRT site), (v) a selectable marker (e.g., dhfr) and (vi) a second polyadenylation signal (e.g., an SV40 E polyA). The term “operably linked” refers to a juxtaposition wherein the components are in a relationship permitting them to function in their intended manner (e.g., functionally linked). Thus, for example, a promoter/enhancer operably linked to a polynucleotide of interest is associated with the latter in such a way that expression of the polynucleotide of interest is achieved under conditions that are compatible with the activation of expression from the promoter/enhancer.

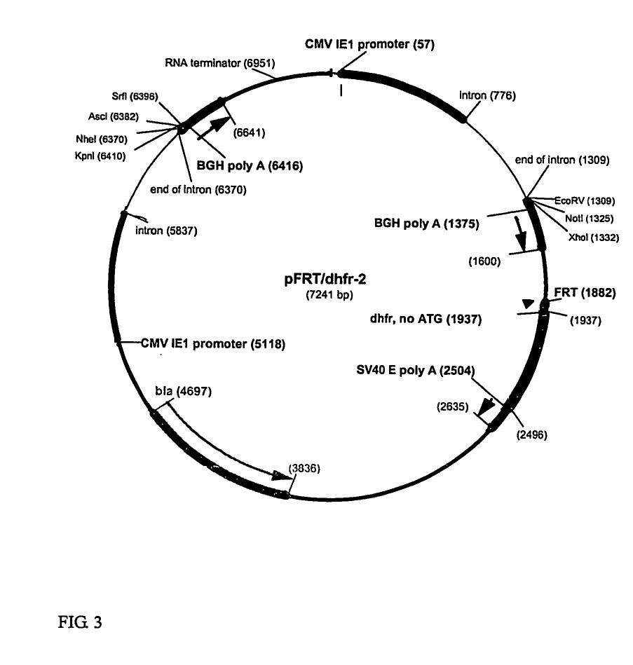

In a one embodiment of the invention, the recombination cassette includes a sequence as set forth in SEQ ID NO:1 from about nucleotide 1 to about nucleotide 2704 (e.g., from about 1 to 2700, to 2701, to 2702, to 2703, to 2705, to 2706, to 2707, or to 2708). As another example, the recombination cassette comprises a sequence from about nucleotide 1 to 2635 (e.g., from about 1 to 2633, to 2634, to 2636, or to 2637) of SEQ ID NO:2. The recombination cassette set forth from nucleotide 1 to 2704 or 1 to 2635 of SEQ ID NO:1 or 2, respectively, includes a number of distinct domains such as a CMV IE1 promoter/enhancer region having a sequence as set forth from about x₁ to about x₂ of SEQ ID NO:1 or 2, wherein x₁ is a nucleotide from position 1 to position 70 and x₂ is a nucleotide from position 770 to position 780 (e.g., from about position 63 to about position 776 of SEQ ID NO:1 or 2). Another domain of the recombination cassette includes a variable length intervening sequence (VLIVS) containing a splice donor and a splice acceptor site. The VLIVS can be at least 50 bp in length (e.g., at least 100, 150, 200, or 250 bp in length) and can include splice donors and acceptors from any source known in the art. See, e.g., Varani et al., Annu Rev Biophys Biomol Struct 27:407-45 (1998) and Koning, Eur J Biochem 219:2542 (1994). A suitable intervening domain can include all of intron A of a CMV genome of any strain or may include a smaller fragment comprising a 5′ sequence containing a splice donor site ligated to a 3′ sequence containing a splice acceptor site. For example, the VLIVS includes nucleotides from about x₃ to about x₄ of SEQ ID NO:1, wherein x₃ is a nucleotide at a position from 770-780 and x₄ is a nucleotide at a position from 1300-1310 (e.g., 776-1304 of SEQ ID NO:1). As another example, the VLIVS includes nucleotides from about x₃ to about x₄ of SEQ ID NO:2, wherein x₃ is a nucleotide from 770-780 and x₄ is a nucleotide from 1300-1310 (e.g., 776-1309 of SEQ ID NO:2). The intervening sequence following the CMV IE1 promoter/enhancer can vary in size as much as 317 nucleotides from that present in SEQ ID NO:1 or 2. A multiple cloning site may be present after (i.e., downstream of) the VLIVS region (e.g., nucleotides 1310-1418 of SEQ ID NO:1 includes NH3I, BamHI, KpnI, EcoRI, PmeI, PstI, EcoRV, NotI, XhoI, ApaI, and PmeI sites; nucleotides 1309-1332 of SEQ ID NO:2 includes EcoRV, NotI, XhoI sites). Different or additional restriction sites may be engineered in the recombination cassette using techniques known to those of skill in the art. For example, with reference to FIG. 3, there is depicted two multiple cloning sites (see, e.g., the cloning sites beginning at about 1309 and 6370) adding the ability to clone in multiple related or unrelated polynucleotides. This vector comprising dual cassettes, as depicted in FIG. 3, also has a terminator between the 2 cassettes.

Furthermore, one of skill in the art will recognize that it is possible to substitute add or delete 1 or more nucleotides from the ends of particular domains without departing from the functionality of the domain and/or the cassette and/or vector as a whole. For example, a variation of from 1 to 10 nucleotides at either end of any one of the domains identified herein will likely comprise a functional domain for the intended purpose of the domain, cassette, and/or vector of the invention.

The recombination cassette further includes a polyA signal domain. The polyA signal domain can be derived from human sources (e.g., human growth hormone (hGH polyA)), or from bovine (e.g., bovine growth hormone (BGH polyA)) or other animal sources. The polyA signal domain can be derived from an hGH gene, which can vary in its 3′UTR sequence, e.g., from allele to allele. One allele of the hGHv gene is described in GenBank Accession No. K00470 (SEQ ID NO:3). An example of a BGH polyA signal domain includes the sequence as set forth from about nucleotide 1143 to about 1668 of SEQ ID NO:1, and from about nucleotide 1375 to about 1600 of SEQ ID NO:2. Non-naturally occurring variants of the polyA signal domain may be made by mutagenesis techniques, including those applied to polynucleotides, cells or organisms. A polyA signal domain variant from a hGH gene includes a polyA signal domain that varies from a wild-type hGH polyA signal domain yet retains the ability to signal transcriptional termination and/or stabilize mRNA. For example, the polyadenylation signal domain may include an hGHv polyadenylation signal domain sequence. Any polyA signal domain that includes a contiguous nucleotide sequence of at least 100 nt (e.g., at least 200, 300, 400, 500, or 600 nt), including the canonical AATAAA site, of an hGHv gene is included.

In addition, the invention encompasses sequences that vary from the foregoing sequences by up to 8% (e.g., have 92% identity to SEQ ID NO:3 or a distinct domain thereof). For example, a polynucleotide having 95% identity to nucleotides 1-2704 of SEQ ID NO:1 or 1-2635 of SEQ ID NO:2 is included within the invention.

In another aspect of the invention a vector comprising a recombination cassette is provided. As used herein, a “vector” is a nucleic acid molecule (either DNA or RNA) capable of autonomous replication upon introduction into a recipient cell (e.g., a bacterial cell or a mammalian cell such as a CHO cell). Plasmids and viruses are examples of vectors. The process of “expression” from an expression vector is well known, and includes the use of cellular enzymes and processes to produce an expression product from a polynucleotide of interest. Expression vectors are vectors that are capable of mediating the expression of a cloned polynucleotide in a host cell, which may or may not be the same type of cell used for replication or propagation of the vector. Many mammalian expression vectors can be propagated in common bacteria (recipient cell) but express the polynucleotide of interest in mammalian cells (host cell) and not in bacteria.

The vectors of the invention include: a cloning site for receiving a polynucleotide of interest; transcription regulatory elements (e.g., CMV IE1 promoter/enhancer regions) sufficient to permit transcription of a polynucleotide inserted into the cloning site in a host cell; translation elements sufficient to permit translation of an RNA transcript of said polynucleotide in a host cell and (if desired) replication elements sufficient to permit the replication of said vector in a host cell or another recipient cell used for propagation of the vector. The vectors of the invention are capable of mediating such expression transiently or stably in host cells (e.g., by homologous recombination within the host cell genome).

In a specific embodiment a vector of the invention includes (1) a sequence as set forth in SEQ ID NO:1 or 2; (2) a sequence that is complementary to the sequence as set forth in SEQ ID NO:1 or 2; (3) a sequence that is at least 80% (or at least 90%; 95%; 98%, or 99%) identical to SEQ ID NO:1 or 2 or their complements; or (4) a sequence comprising SEQ ED NO:1 or 2 from about nucleotide 1 to about nucleotide 2704 or 2635, respectively, and comprising a polynucleotide of interest and/or a selectable marker.

A vector of the invention comprises SEQ ID NO:1 or 2, or one or more of the following domains so long as it contains an FRT site. For example, a CMV IE1 promoter/enhancer region having a sequence as set forth from about x₁ to about x₂ of SEQ ID NO:1, wherein x₁ is a nucleotide from 1-70 and x₂ is a nucleotide from 770-780 (e.g., from about 1 to 776 of SEQ ID NO:1) is present in the vector. In another aspect of the invention a CMV IE1 promoter/enhancer region having a sequence as set forth from about x₁ to about x₂ of SEQ ID NO:2, wherein x₁ is a nucleotide from 1-60 and x₂ is a nucleotide from 770-780 (e.g., from about 1 to 776 of SEQ ID NO:1) is present in the vector. Another domain of an expression vector of the invention includes a variable length intervening sequence (VLIVS) containing a splice donor and splice acceptor site. For example, the VLIVS includes nucleotides from about x₃ to about x₄ of SEQ ID NO:1, wherein X₃ is a nucleotide from 770-780 and x₄ is a nucleotide from 1300-1310 (e.g., 776-1304 of SEQ ID NO:1). As another example, the VLIVS includes nucleotides from about x₃ to about x₄ of SEQ ID NO:2, wherein x₃ is a nucleotide from 770-780 and x₄ is a nucleotide from 1300-1310 (e.g., 776-1309 of SEQ ID NO:2). A multiple cloning site may be present after (i.e., downstream of) the VLIVS region (e.g., nucleotides 1310-1418 of SEQ ID NO:1 includes NH3I, BamHI, KpnI, EcoRI, PmeI, PstI, EcoRV, NotI, XhoI, ApaI, and PmeI sites; nucleotides 1309-1332 of SEQ ID NO:2 includes EcoRV, NotI, XhoI sites). Different or additional restriction sites may be engineered in the expression vector using techniques known to those of skill in the art. The expression vector further includes a polyA signal domain.

The polyA signal domain can be derived from human sources (e.g., human growth hormone (hGH polyA), or from bovine (e.g., bovine growth hormone (BGH polyA)) or other animal sources. The polyA signal domain can be derived from an hGHv gene, which can vary in its 3′UTR sequence, e.g., from allele to allele. One allele of the hGHv gene is described in GenBank Accession No. K00470 (SEQ ID NO:3). An example of a BGH polyA include the sequence as set forth from nucleotide 1143 to 1668 of SEQ ID NO:1, and from nucleotide 1375 to 1600 of SEQ ID NO:2. Also present in a vector of the invention is one or more selectable markers.

The recombination cassettes and vectors of the invention have distinctive advantages over prior recombination cassettes and vectors. For example, the cassettes and vectors of the invention allow stable integration of a polynucleotide of interest into a host cell comprising one or more FRT sites and utilizing a dhfr selectable marker that is auxotrophic in nature and allows for simple and efficient selection of transformed host cells. Selection of successfully transfected cell lines usually relies on an integrated selectable marker that confers resistance to cytotoxic drugs (e.g., antibiotic resistance). Non-auxotrophic selectable markers confer resistance to substances that would normally kill an organism (e.g., a cell). When this cytotoxic substance is applied to the organism only those with the selectable marker will survive. Thus, typical selectable markers require that an exogenous substance must be added in order to select a cell that is resistant. Proper concentrations of the cytotoxic substance must be added to the culture requiring additional effort on the part of the technician or researcher. For example, if too much cytotoxic substance is added, then organisms that include a selectable marker may also be killed thereby reducing transfection/transformation efficiency and yield. In contrast, the invention provide a selectable marker wherein a cytotoxic substance is not added, but rather the cells having the selectable marker (i.e., dhfr) are capable of growing in a defined medium lacking a specific additive that is necessary for organisms lacking the selectable marker to grow. Dihydrofolate reductase (DHFR) is an NADPH-requiring enzyme (EC 1.5.1.3) which catalyses the synthesis of tetrahydrofolate, a metabolite essential for the synthesis of dTMP, glycine, and purines. In the absence of a polynucleotide encoding DHFR, a cell must be grown on medium containing dTMP, glycine, and/or purines. Where the selectable marker, DHFR, is present in a cell, the exogenous purines can be removed and those cells containing the DHFR marker will continue to grow and proliferate whereas those lacking DHFR will die. Accordingly, the invention provides less effort in selecting organisms (e.g., cells) that have been transfected/transformed.

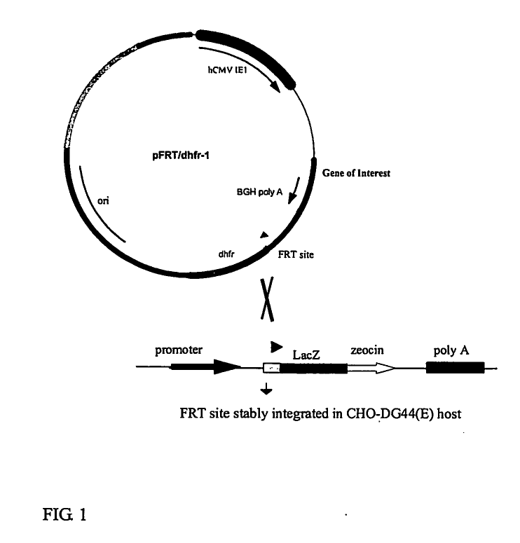

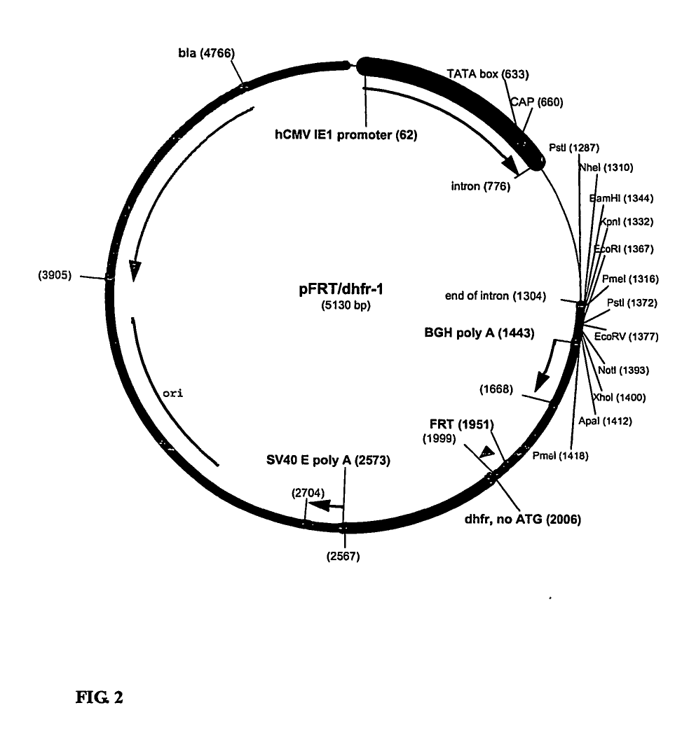

The FLP system of the invention allows for stable integration and expression of a polynucleotide of interest in any desired mammalian host cell at a specific genomic location. An FLP host cell line is established by transfecting a single FLP recombination target (FRT) domain into the genome of a chosen host cell line; it is this resulting host cell line that is then used for subsequent FLP transfections/recombinations. Once the host cell line is generated, any polynucleotide of interest can be stably integrated into the host cell genome via FLP recombinase- mediated DNA recombination at the FRT site, see FIG. 1. A polynucleotide of interest can effectively be “swapped in” to the host genome, always in the identical location and orientation. Since all transfected cells will have the polynucleotide of interest integrated into the same genomic location, the isolation of clonal cell lines is obviated because all of the transfected cells are genetically identical.

The invention also provides a host cell line that was generated by transfecting Chinese Hamster Ovary (CHO)-DG44 cells with a recombination target site (e.g., an FRT recombination site). This CHO-DG44 cell line lacks a functional dihydrofolate reductase (dhfr) gene, thus requiring exogenous glycine, purines, and thymidine (a pyrimidine) for growth. Such a cell line should be maintained in culture medium supplemented with nucleosides. Upon transfection with a recombination cassette/vector of the invention, which contains a functional dhfr gene, selection is accomplished by culturing cells in medium free of purines and thymidine. With the establishment of the host cell line, the generation of stable cell lines expressing a polynucleotide(s) of interest can be very rapid.

The invention further provides a recombination system comprising a recombination cassette and/or vector of the invention and a host cell comprising a FRT site. In one aspect of the invention the host cell is a CHO cell or a CHO-derived cell comprising a FRT site recombinantly inserted into the genome of the host cell. This system (e.g., the CHO or CHO-derived cell and the recombination cassette/vector) provides a powerful tool for generating between 10 and 40 mg/L of a recombinant protein in a CHO host in less than 6 weeks (e.g., having an ICA of 15×10⁷ (cell days)/ml during a 10-day bioreactor run). Furthermore, minimal work is required to establish these stable cell lines.

The methods and compositions of the invention allow for easy integration of a polynucleotide of interest at a predetermined endogenous polynucleotide target site in a host cell. The endogenous polynucleotide target site can either be a naturally occurring polynucleotide (i.e., a polynucleotide that occurs in the genome of the host and is not recombinantly inserted) or may be a polynucleotide that has been previously engineered into the host organism to effectuate selection, expression, or recombination in the host organism. As used herein, the terms “predetermined endogenous polynucleotide target” and “predetermined target” refer to polynucleotide sequences contained in a target cell. Such a predetermined target comprises, for example, chromosomal sequences (e.g., structural genes, intronic sequences, 5′ or 3′ noncoding sequences, regulatory sequences including promoters and enhancers, recombinatorial hotspots, repeat sequences, integrated proviral sequences, hairpins, palindromes), and episomal or extrachromosomal sequences (e.g., replicable plasmids or viral or parasitic replication intermediates) including chloroplast and mitochondrial DNA sequences. By “predetermined” or “pre-selected” it is meant that the target sequence may be selected based upon predicted sequence information, and is not constrained to specific sites recognized by certain site-specific recombinases (e.g., FLP recombinase or CRE recombinase). In some embodiments, the predetermined endogenous polynucleotide target will be other than a naturally occurring germine polynucleotide (e.g., an exogenous polynucleotide, parasitic, mycoplasmal or viral sequence). An exogenous polynucleotide is a polynucleotide that is transferred (i.e., by recombinant molecular biology techniques) into a host cell. For example, exogenous polynucleotides that are microinjected or transfected into a cell are exogenous polynucleotides. The term “naturally-occurring” as used herein as applied to an object refers to the fact that the object can be found in nature. For example, a polynucleotide that is present in an organism (including a virus) that can is be isolated from a source in nature and that has not been modified by man is naturally-occurring.

A recombination domain in the recombination cassette/vector of the invention directs the cassette/vector to a specific chromosomal location within the genome of a host by virtue of the homology that exists between the recombination domain and the corresponding predetermined endogenous polynucleotide target and introduces the desired genetic modification by a process referred to as “homologous recombination”.

“Homologous” or “homology” means two or more nucleic acid sequences that are either identical or similar enough (e.g., 97% or more identical) that they are able to hybridize to each other or undergo intermolecular exchange. The percentage of sequence identity is calculated excluding small deletions or additions which total less than 25 percent of the reference sequence. The reference sequence may be a subset of a larger sequence, such as a portion of a gene or flanking sequence, or a repetitive portion of a chromosome. However, the reference sequence is at least 12-18 nucleotides long, at least about 30 nucleotides long, and can be at least about 50 to 100 nucleotides long. In general, recombination efficiency increases as the length and/or percentage of homology between the recombination domain and the predetermined endogenous polynucleotide target increases.

The terms “identical” or percent “identity,” in the context of two or more nucleic acid molecules, refer to two or more sequences or subsequences that are the same or have a specified percentage of nucleotides that are the same, when compared and aligned for maximum correspondence over a comparison window, as measured using a comparison algorithm or by manual alignment and visual inspection. This definition also refers to the complement of a sequence (e.g., the complement of a sequence as set forth in SEQ ID NO:1 or a fragment thereof comprising a recombination cassette). For example, the recombination cassette and fragments thereof include those with a nucleotide sequence identity that is at least about 80%, about 90%, and about 95%, about 97%, about 98% or about 99% identical to a defined portion of SEQ ID NO:1 (e.g., nucleotides 1-719, 1-1254, and the like, of SEQ ID NO:1). Thus, if a sequence has the requisite sequence identity to the full sequence of SEQ ID NO:1 or a domain thereof then it can also function as a recombination cassette or domain of the invention, respectively.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identity for the test sequence(s) relative to the reference sequence, based on the designated or default program parameters. A “comparison window”, as used herein, includes reference to a segment of any one of the number of contiguous positions selected from the group consisting of from 25 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Various algorithms are known in the art and include, e.g., the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, PILEUP, BESTFIT, FASTA, and TFASTA in the Wisconsin Genetics Software Package, Genetics Computer Group, 575 Science Dr., Madison, Wis.), or by manual alignment and visual inspection.

For purposes of determining percent sequence identity of the described invention (i.e., substantial similarity or identity) the BLAST algorithm is used, which is described in Altschul, J. Mol. Biol. 215:403-410, 1990. Software for performing BLAST analyses is publicly available through the National Center for Biotechnology Information (on the World Wide Web at ncbi.nlm.nih.gov/). This algorithm involves first identifying high scoring sequence pairs (HSPs) by identifyig short words of length W in the query sequence, which either match or satisfy some positive-valued threshold score T when aligned with a word of the same length in a database sequence. “T” is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues, always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment and include the following parameters for nucleotide comparison: a wordlength (W) of 11, an expectation (L) of 10, M=5, N=4. For amino acid sequences, the BLASTP program uses a wordlength (W) of 3, an expectation (E) of 10, and the BLOSUM62 scoring matrix (see, e.g., Henikoff, Proc. Natl. Acad. Sci. USA 89:10915, 1989).

The BLAST algorithm also performs a statistical analysis of the similarity between two sequences (see, e.g., Karlin, Proc. Nat'l. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. A nucleic acid is considered similar to a reference sequence if the smallest sum probability is less than 0.1. For example, it can be less than about 0.01, or less than about 0.001.

Also included in the invention are polynucleotides that specifically hybridize to a polynucleotide sequence as set forth in SEQ ID NO:1 or 2 or a domain thereof. The phrase “selectively (or specifically) hybridizes to” refers to the binding, duplexing, or hybridizing of a molecule to a particular reference polynucleotide under stringent hybridization conditions. The phrase “stringent hybridization conditions” refers to conditions under which a probe will primarily hybridize to its target subsequence in a complex mixture of nucleic acid, but to no other sequences. Stringent conditions are sequence-dependent and will be different in different circumstances, e.g., depending on the length of the probe. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength and pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts), at pH 7.0 to 8.3 and the temperature is at least about 30° C. for short probes (e.g., 10 to about 50 nucleotides) and at least about 60° C. for long probes (e.g., greater than about 50 nucleotides). Stringent conditions may also be achieved with the addition of destabilizing agents such as formamide. For selective or specific hybridization, a positive signal (e.g., identification of a nucleic acid of the invention) is about 2 times background hybridization. For the purpose of this invention, moderately stringent hybridization conditions mean that hybridization is performed at about 42° C. in a hybridization solution containing 25 mM KPO₄ (pH 7.4), 5×SSC, 5× Denhart's solution, 50 μg/mL denatured, sonicated salmon sperm DNA, 50% formamide, 10% Dextran sulfate, and 1-15 ng/mL probe, while the washes are performed at about 50° C with a wash solution containing 2X SSC and 0.1% sodium dodecyl sulfate. Highly stringent hybridization conditions mean that hybridization is performed at about 42° C. in a hybridization solution containing 25 mM KPO₄ (pH 7.4), 5×SSC, 5× Denhart's solution, 50 μg/mL denatured, sonicated salmon sperm DNA, 50% formamide, 10% Dextran sulfate, and 1-15 ng/mL probe, while the washes are performed at about 65° C. with a wash solution containing 0.2×SSC and 0.1% sodium dodecyl sulfate.

The methods and compositions of the invention find use in the modification of a host cell by the insertion into, deletion of, or replacement of an endogenous polynucleotide via homologous recombination using a cassette/vector of the invention.

A recombination cassette/vector of the invention can comprise a selectable marker. A “marker” or a “selectable marker” is a selection marker that allows for the isolation of rare transfected cells expressing the marker from the majority of treated cells in population. Specific examples of selectable markers are those that encode proteins that confer resistance to cytostatic or cytocidal drugs, such as the DHFR protein, which confers resistance to methotrexate (Wigler et al., Proc. Natl. Acad. Sci. USA 77:3567 (1980); O'Hare et al., Proc. Natl. Acad. Sci. USA 78:1527 (1981)); the GPF protein, which confers resistance to mycophenolic acid (Mulligan & Berg, Proc. Natl. Acad. Sci. USA 78:2072 (1981)); the neomycin resistance marker, which confers resistance to the aminoglycoside G-418 (Colberre-Garapin et al., J. Mol. Biol. 150:1 (1981)); the Hygro protein, which confers resistance to hygromycin (Santerre et al., Gene 30:147 (1984)); and the Zeocin™ resistance marker (available commercially from Invitrogen). In addition, the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:2026 (1962)), and adenine phosphoribosyltransferase (Lowy et al., Cell 22:817 (1980)) can be employed in tk-, hgprt- or aprt-cells, respectively. Other selectable markers encode puromycin N-acetyl transferase or adenosine deaminase.

Of particular advantage is the dhfr marker. DBFR is an enzyme that is required for pyrimidine synthesis. Cells that lack a functional DHFR or do not express DoFR require pyrimidines to grow. A host cell that is dhfr- can be transfected with a dhfr vector is thereby restoring the ability to synthesize pyrimidines. When medium containing exogenous pyrimidines is removed, only those cells comprising the exogenously provided dhfr gene will survive. In contrast, markers that select based upon their ability to grow in the presence of cytotoxic drugs are more difficult to select. For example, where a cell is transfected with a “resistance gene,” various concentrations of cytotoxic drugs are provided to select cells that comprise the resistance gene. In some cases, too much of the cytotoxic drug may be added or not enough, in which case the selection is inefficient and/or unreliable.

SEQ ID Nos: 1 and 2 include a dihydrofolate reductase (dhfr) gene (e.g., comprising a sequence from about nucleotide 2007 to about nucleotide 2567 of SEQ ID NO:1; and from about nucleotide 1939 to about nucleotide 2499 of SEQ ID NO:2). A recombination cassette/vector of the invention may include additional promoter/enhancer elements and regulatory regions (e.g., polyadenylation domains). Such additional regulatory elements and polyadenylation domains may flank (e.g., be immediately adjacent to, 5′ and 3′ of) a selectable marker or polynucleotide of interest. The dhfr gene in these vectors is flanked by an SV40 polyadenylation region (e.g., about nucleotide 2568 to about nucleotide 2704 and about nucleotide 2500 to about nucleotide 2635 of SEQ ID NO:2, respectively).

A recombination cassette and/or a vector of the invention comprises a recombination domain of at least about 10 to 100 nucleotides, typically at least about 20 to 100 nucleotides long, but can be longer (e.g., at least about 250 to 500 nucleotides long, or about 500 to 2000 nucleotides long, or longer). The length of the recombination domain will be based, in part, upon the homology with a predetermined endogenous polynucleotide target. Accordingly, the length of homology may be selected at the discretion of the practitioner on the basis of the sequence composition and complexity of the predetermined endogenous polynucleotide target sequence(s) and guidance provided in the art. The recombination domain has at least one sequence that substantially corresponds to, or is substantially complementary to, a predetermined endogenous polynucleotide (e.g., a DNA sequence of a polynucleotide located in a target host cell, such as a chromosomal, mitochondrial, chloroplast, viral, episomal, or mycoplasmal polynucleotide). Such recombination domain nucleotide sequences serve as templates for homologous pairing with the predetermined endogenous polynucleotide target(s). In targeting a polynucleotide of interest in a vector to a host cell genome, the regions of homology are typically located at or near the 5′ and/or 3′ end(s) of the polynucleotide of interest (Berinstein et al. (1992) Molec. Cell. Biol. 12: 360, which is incorporated herein by reference). Without wishing to be bound by any particular theory, it is believed that the addition of recombinases permits efficient targeting with a recombination domain nucleotide sequence having short (i.e., about 10 to 1000 basepair long) segments of homology. In the invention, the recombination domain is an FRT site.

Typically the recombination domain nucleotide sequence will have high degree of homology to the predetermined polynucleotide endogenous target, and will typically be identical. Typically, the recombination domain nucleotide sequence of the invention is about 10 to 35 nucleotides long, but can be about 20 to 100 nucleotides long, or about 100 to 500 nucleotides long, although the degree of sequence homology between the recombination domain and the predetermined endogenous polynucleotide target will determine the optimal and minimal lengths (e.g., G-C rich sequences are typically more thermodynamically stable and will generally require shorter recombination domain length). Therefore, both recombination domain length and the degree of sequence homology can be determined with reference to a particular predetermined sequence.

A recombination cassette of the invention and/or a vector of the invention are introduced into a host cell harboring a predetermined endogenous polynucleotide target, generally with at least one recombinase protein (e.g., an Flp recombinase). Under some circumstances, the recombination cassette or vector is incubated with Flp or other recombinase prior to introduction into a host cell, so that the recombinase protein(s) may be “loaded” onto the recombination cassette or recombination vector.

Recombinases are proteins that, when included with a polynucleotide of interest comprising a recombination domain, provide a measurable increase in the recombination frequency and/or localization frequency between the polynucleotide of interest and a predetermined endogenous polynucleotide target. Thus, in one embodiment, increases in recombination frequency 10 to a 1000 fold may be achieved.

A recombinase is a member of a family of RecA-like recombination proteins all having essentially all or most of the same functions, particularly: (i) the recombinase protein's ability to properly bind to and position a polynucleotide of interest comprising a recombination domain on its homologous target, and (ii) the ability of recombinase protein/targeting polynucleotide complexes to efficiently find and bind to complementary endogenous sequences. The best-characterized recA protein is from E. coli. In addition to the wild-type E. Coli protein, a number of mutant recA-like proteins have been identified (e.g., recA803; see Madiraju et al., Proc. Natl. Acad. Sci. USA 85(18):6592 (1988); Madiraju et al., Biochem. 31:10529 (1992); Lavery et al., J. Biol. Chem. 267:20648 (1992)). Further, many organisms have recA-like recombinases with strand-transfer activities (e.g., Fugisawa et al., Nucl. Acids Res. 13: 7473 (1985); Hsieh et al., Cell 44: 885 (1986); Hsieh et al., J. Biol. Chem. 264: 5089 (1989); Fishel et al., Proc. Natl. Acad. Sci. USA 85: 3683 (1988); Cassuto et al., Mol. Gen. Genet. 208: 10 (1987); Ganea et al., Mol. Cell Biol. 7: 3124 (1987); Moore et al., J. Biol. Chem. 19: 11108 (1990); Keene et al., Nucl Acids Res. 12: 3057 (1984); Kimeic, Cold Spring Harbor Symp. 48: 675 (1984); Kimeic, Cell 44: 545 (1986); Kolodner et al., Proc. Natl. Acad. Sci. USA 84: 5560 (1987); Sugino et al., Proc. Natl. Acad. Sci. USA 85: 3683 (1985); Halbrook et al., J. Biol. Chem. 264: 21403 (1989); Eisen et al., Proc. Natl. Acad. Sci. USA 85: 7481 (1988); McCarthy et al., Proc. Natl. Acad. Sci. USA 85: 5854 (1988); Lowenhaupt et al., J. Biol. Chem. 264: 20568 (1989), which are incorporated herein by reference. Examples of such recombinase proteins include, but are not limited to: recA, recA803, uvsX, and other recA mutants and recA-like recombinases (Roca, A. I. Crit. Rev. Biochem. Molec. Biol. 25: 415 (1990)), sep1 (Kolodner et al., Proc. Natl. Acad. Sci. USA 84:5560 (1987); Tishkoffet al., Molec. Cell. Biol. 11:2593, (1991)), RuvC (Dunderdale et al., Nature 354: 506 (1991)), DST2, KEM1, XRN1 (Dykstra et al., Molec. Cell. Biol. 11:2583 (1991)), STP.alpha./DST1 (Clark et al., Molec. Cell. Biol. 11:2576 (1991)), HPP-1 (Moore et al., Proc. Natl. Acad. Sci. USA 88:9067 (1991)), other target recombinases (Bishop et al., Cell 69: 439 (1992); Shinohara et al., Cell 69: 457 (1992)), all incorporated herein by reference. RecA may be purified from E. coli, such as E. coli strains JC12772 and JC15369 (which can be purchased commercially). These strains contain the recA coding sequences on a “runaway” replicating plasmid vector present at a high copy numbers per cell. The recA803 protein is a high-activity mutant of wild-type recA. The art teaches several examples of recombinase proteins, for example, from Drosophila, yeast, plant, human, and non-human mammalian cells, biological properties similar to recA (i.e., recA-like recombinases), such as Rad51 from mammals and yeast and Pk-rec from the hyperthermophilic archaeon Pyrococcussp (see Rashid et al., Nucleic Acid Res. 25(4):719 (1997), hereby incorporated by reference). The recombinase may actually be a complex of proteins. Included within the definition of a recombinase are portions or fragments of recombinases that retain recombinase biological activity, as well as variants or mutants of wild-type recombinases that retain biological activity, such as the E. coli recA803 mutant with enhanced recombinase activity.

RecA forms homologous joints between homologous sequences, and is implicated as mediating a homology search process between an exogenous polynucleotide strand (e.g., a polynucleotide of interest comprising a recombination domain) and an endogenous polynucleotide strand (e.g., a predetermined polynucleotide target), producing relatively stable heteroduplexes at regions of high homology. Accordingly, recombinases can drive the homologous recombination reaction between strands that are significantly but not perfectly homologous. Thus, a recombination cassette/vector may be used to introduce nucleotide substitutions, insertions and deletions into an endogeneous DNA sequence, and thus the corresponding amino acid substitutions, insertions and deletions in proteins expressed from the endogeneous DNA sequence.

In one embodiment, recA or rad51 is used as the recombinase. For example, recA protein is typically obtained from bacterial strains that overproduce a wild-type E. coli recA protein or mutant recA803 protein. Alternatively, recA protein can be purchased from, for example, Pharmacia (Piscataway, N.J.).

FLP recombinase is a protein that catalyzes a site-specific recombination reaction. The FLP protein has been cloned and expressed in E. coli (see, e.g., Cox, Proc. Natl. Acad. Sci. USA., 80:4223-4227, 1983), and has been purified to near homogeneity (see, e.g., Meyer-Leon et al., Nucl. Acids Res., 15:6469-6488, 1987). FLP recombinases are commercially available or can be obtained by those of skill in the art from the genus Saccharomyces, for example.

An FRT site has been identified as comprising two 13 base-pair repeats, separated by an 8 base-pair spacer: for example, a sequence comprising 5′-GAAGTTCCTATTCTCTAGAAAGTATAGGAACTTC-3′ (SEQ ID NO:1 from nucleotide 1966 to 1999 and SEQ ID NO:2 from 1882 to 1937; the italicized sequence representing the spacer). The nucleotides in the spacer region can be replaced with any other combination of nucleotides, so long as the two 13 base-pair repeats are separated by at least 8 nucleotides. The actual nucleotide sequence of the spacer is not critical, although those of skill in the art recognize that, for some applications, it is desirable for the spacer to be asymmetric, while for other applications, a palindromic spacer can be employed. Generally, the spacers present in the recombination domain and in the FRT site present in the host cell will be identical with one another. A recombination domain of the invention and a FRT site in a host cell can comprise a sequence as set forth from nucleotide 1966 to nucleotide 1999 of SEQ ID NO:1 and nucleotide 1898 to nucleotide 1931 of SEQ ID NO:2.

Recombinase mediated processes are further described in the following publications: WO 00/63365; WO 99/60108; WO 00/56872; WO 99/37755; U.S. Pat. Nos. 5,948,653, 6,074,853, 5,763,240, 5,929,043,and 5,989,879, all of which are incorporated by reference herein in their entirety. It is understood that the compositions and methods of the invention utilize recombinases such as those described herein as well as others known to those of skill in the art.

As discussed above, the recombination cassette and vector comprise regulatory elements. In one aspect of the invention these promoter and optionally enhancer elements are from any strain of cytomegalovirus, such as described herein or in references such as U.S. Pat. No. 5,658,759, the disclosure of which is incorporated herein by reference. For example, suitable CMV immediate early promoter regions useful in the recombination cassettes of the invention can be obtained from the CMV-promoted β-galactosidase expression vector, CMVβ (MacGregor et al., Nucl. Acids Res. 17:2365 (1989)).

The recombination cassette may be used in the form of a naked nucleic acid construct. Alternatively, the recombination cassette may be introduced as part of a nucleic acid vector (e.g., a recombination vector such as those described above). Such vectors include plasmids and viral vectors.

The term “polynucleotide of interest” is intended to cover nucleic acid molecules that are capable of being transcribed. The molecule may be in the sense or antisense orientation with respect to a promoter. Antisense constructs can be used to inhibit the expression of a gene in a cell according to well-known techniques. The polynucleotide of interest may include a heterologous polynucleotide. A heterologous polynucleotide typically originates from a foreign species compared to the regulatory element with which it is operably linked in the recombination cassette or vector or if originated from the same source, is modified from its original form. Therefore, a heterologous polynucleotide operably linked to a promoter is from a source different from that from which the promoter was derived, or, if originated from the same source, is modified from its original form. Modification of the heterologous polynucleotide may occur, e.g., by treating the DNA with a restriction enzyme to generate a DNA fragment that is capable of being operably linked to the promoter thereby modifying the polynucleotide from its original form. Site-directed mutagenesis is also useful for modifying a heterologous polynucleotide. Heterologous polynucleotides may also include marker genes (e.g., encoding β-galactosidase or green fluorescent protein) or genes whose products regulate the expression of other genes. Thus polynucleotides that serve as templates for mRNA, tRNA and rRNA are included within this definition. The heterologous gene may be any allelic variant of a wild-type gene, or it may be a mutant gene. mRNA will optionally include some or all of 5′ and/or 3′ transcribed but untranslated flanking regions naturally, or otherwise, associated with the translated coding sequence.

The polynucleotide of interest may optionally further include the associated transcriptional control elements normally associated with the transcribed molecules, for example transcriptional stop signals, polyadenylation domains and downstream enhancer elements. The polynucleotide of interest can encode or serve as template for a therapeutic product, which can for example be a peptide, polypeptide, protein, or ribonucleic acid. The polynucleotide of interest is typically a DNA sequence (such as cDNA or genomic DNA) coding for a polypeptide product such as enzymes (e.g. β-galactosidase); hormones; cytokines; interleukins; interferons; TNF; growth factors (e.g. IGF-1); soluble receptor molecules (e.g., soluble TNF receptor molecules); neurotransmitters or their precursors; trophic factors such as BDNF, CNTF, NGF, IGF, GMF, aFGF, bFGF, NT3 and NT5; apolipoproteins such as ApoAI and ApoAIV; dystrophin or a minidystrophin; tumor-suppressing proteins such as p53, Rb, Rap1A, DCC and k-rev; factors involved in coagulation such as factors VII, VIII and IX; or alternatively all or part of a natural or artificial immunoglobulin (e.g. Fab and ScFv, or the light or heavy chain of a cloned IgG).

A polynucleotide of interest may also include a template for generation of an antisense molecule, the transcription of which in a target cell enables gene expression or the transcription of cellular mRNAs to be controlled. Such molecules can, for example, be transcribed in a target cell into RNAs complementary to cellular mRNAs and can thus block their translation into protein, according to techniques known in the art. In particular, antisense molecules can be used to block translation of inflammatory or catabolic cytokines in the treatment of arthritis and tissue loss caused by these cytokines.

The polynucleotide of interest typically will encode a polypeptide of diagnostic or therapeutic use. The polypeptide may be produced in bioreactors in vitro using various host cells (e.g., COS cells or CHO cells or derivatives thereof) containing the recombination cassette of the invention.

By a therapeutic use is meant a use that may provide relief from a disease or disorder, cure a disease or disorder, and/or ameliorate the severity of a disease or disorder. A diagnostic use includes using molecules capable of determining or providing information regarding a cause or relationship of a molecule to a disease process or determining the presence or absence of a disease or disorder. A diagnostic agent does not directly contribute to the amelioration of the disease or disorder.

A polynucleotide of interest may also encode an antigenic polypeptide for use as a vaccine. Polynucleotides that encode antigenic polypeptides are derived from pathogenic organisms such as, for example, a bacterium or a virus. For example, antigenic polypeptides include antigenic determinants present in a polypeptide of a pathogenic organism. Accordingly, vaccines for such organisms that cause, for example, viral haemorrhagic septicemia, bacterial kidney disease, vibriosis, and furunculosis may be obtained.

As used herein, “isolated,” when referring to a molecule or composition, such as, e.g., a vector or recombination cassette of the invention, or polynucleotide of interest, means that the molecule or composition is separated from at least one other compound, such as a protein, DNA, RNA, or other contaminants with which it is associated in vivo or in its naturally occurring state. Thus, a polynucleotide of interest is considered isolated when it has been isolated from any other component with which it is naturally associated. An isolated composition can be substantially pure. An isolated composition can be in a homogeneous state. It can be dry/lyophilized or in an aqueous solution. Purity and homogeneity can be determined, e.g., using analytical chemistry techniques such as, e.g., polyacrylamide gel electrophoresis (PAGE), agarose gel electrophoresis or high-pressure liquid chromatography (HPLC).

As used herein, “recombinant” refers to a polynucleotide synthesized or otherwise manipulated in vitro (e.g., “recombinant polynucleotide”), to methods of using recombinant polynucleotides to produce products in cells or other biological systems, or to a polypeptide (“recombinant protein”) encoded by a recombinant polynucleotide. Recombinant polynucleotides encompass nucleic acid molecules from different sources ligated into a recombination cassette or vector for expression of, e.g., a fusion protein; or those produced by inducible or constitutive expression of a polypeptide (e.g., a recombination cassette or vector of the invention operably linked to a heterologous polynucleotide, such as a polypeptide coding sequence).

In a typical expression system, production of a polypeptide from a heterologous polynucleotide is either not regulated or is regulated by modulating transcription from a transcriptional promoter operably linked upstream of a polynucleotide that encodes the heterologous polypeptide. However, regulation must also occur properly downstream in order provide proper transcriptional termination and mRNA stability. In one aspect of the invention, a polyadenylation (polyA) signal domain is provided downstream (3′) of a polynucleotide of interest present in a recombination cassette or vector of the invention. In one aspect, an hGHv polyA signal domain is used and includes a sequence derived from the human growth hormone genetic sequence. The hGHv polyadenylation signal domain sequence provides for a strong transcriptional termination and provides increased mRNA stability in eukaryotic cells. This hGHv polyadenylation signal domain provides a distinctive advantage over prior recombination cassettes and/or vectors including those that may utilize a CMV promoter/enhancer.

Translation elements may also be present and are intended to encompass the specialized sequences (such as ribosome binding sites and initiation codons) that are necessary to permit translation of an RNA transcript into protein. Translation elements may also include consensus sequences, leader sequences, splice signals, and the like, that serve to facilitate or enhance the extent of translation, or increase the stability of the expressed product. The vectors of the invention may possess ancillary transcription regions, such as introns, polyadenylation signals, Shine/Dalgamo translation signals and Kozak consensus sequences (Shine et al., Proc. Natl. Acad. Sci. (U.S.A.) 71:1342-1346 (1974); Kozak, Cell 44:283-292 (1986)).

The term “replication elements” is intended to encompass the specialized sequences (such as origins of replication) that are necessary to permit replication of the vector in a recipient cell. In general, such vectors will contain at least one origin of replication sufficient to permit the autonomous stable replication of the vector in a recipient cell.

In a further embodiment, the invention relates to host cells containing the above-described constructs (e.g., the recombination cassette or vector of the invention). The recombination cassette of the invention may be used to recombinantly modify a host cell by transfecting a host cell or transforming a host cell to express a desired polynucleotide of interest. As used herein, the term “recombinantly modified” means introducing a recombination cassette or vector of the invention into a living cell or expression system. Usually, the recombination cassette comprising a polynucleotide of interest is present in a vector (e.g., a plasmid). An expression system includes a living host cell into which a polynucleotide of interest, whose product is to be expressed, has been introduced, as described herein.

Host cells are cells in which a recombination cassette (including a vector comprising a recombination cassette) can be propagated and polynucleotides encoding products can be expressed. A host cell also includes any progeny of the subject host cell or its derivatives. It is understood that all progeny may not be identical to the parental cell since there may be mutations that occur during replication. However, such progeny are included when the term “host cell” is used. Host cells that are useful in the invention include bacterial cells (e.g., E. coli), fungal cells (e.g., yeast cells), plant cells and animal cells. For example, host cells can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (Davis, L., Dibner, M., Battey, I., Basic Methods in Molecular Biology (1986)). As representative examples of appropriate hosts, there may be mentioned: fungal cells, such as yeast; insect cells such as Drosophila S2 and Spodoptera Sf9; animal cells such as CHO, COS or Bowes melanoma; plant cells, and the like. The selection of an appropriate host is deemed to be within the scope of those skilled in the art from the teachings herein.

Host cells for use in the invention include eukaryotic host cells (e.g., mammalian cells). In one aspect of the invention the host cells are mammalian production cells adapted to grow in cell culture. Examples of such cells commonly used in the industry are CHO, VERO, BHK, HeLa, CV1 (including Cos; Cos-7), MDCK, 293, 3T3, C127, myeloma cell lines (especially murine), PC12 and W138 cells. Chinese hamster ovary (CHO) cells are widely used for the production of several complex recombinant proteins, e.g. cytolines, clotting factors, and antibodies (Brasel et al., Blood 88:2004-2012 (1996); Kaufman et al., J. Biol Chem 263: 6352-6362 (1988); McKinnon et al., J Mol Endocrinol 6:231-239 (1991); Wood et al., J. Immunol 145:3011-3016 (1990)). The dihydrofolate reductase (DHFR)-deficient mutant cell lines (Urlaub et al., Proc Natl Acad Sci USA 77:4216-4220 (1980)) are the CHO host cell lines of choice because the efficient DHFR selectable and amplifiable gene expression system allows high level recombinant protein expression in these cells (Kaufman, Meth Enzymol 185:527-566 (1990)). In addition, these cells are easy to manipulate as adherent or suspension cultures and exhibit relatively good genetic stability. CHO cells and recombinant proteins expressed in them have been extensively characterized and have been approved for use in clinical manufacturing by regulatory agencies. In addition, it is contemplated that host cells derived from any of the foregoing cell lines and having a desired phenotype may also be used. For example, a derived host cell includes CHO cells (e.g., the DG44 cell line) that have been selectively cultured for a desired phenotype (e.g., by positive and/or negative selection processes). In one aspect, the CHO cells are adapted to grow in serum free medium and may also or independently be adapted to grown in suspension (see, e.g., Sinacore et al., Biotechnol. Bioengin, 52:518-528 (1996); and Haldankar et al., Biotechnol. Prog. 15:336-346 (1999)). Suspension adapted cells are easier to handle and can achieve higher densities. Serum free adapted cells offer the advantage of ease of purification of a recombinant protein from the cell culture supernatant.

In one aspect of the invention, an expression system for in vitro production of an agent encoded by a polynucleotide of interest is provided. As discussed herein, the polynucleotide of interest can encode a polypeptide of pharmaceutical, medicinal, nutritional, and/or industrial value. For example, the polynucleotide of interest can encode a polypeptide-based drug. Typically such a polypeptide will be expressed as an extracellular product. For example, polypeptides that may be produced using the recombination cassette and/or vector of the invention include but are not limited to a Flt3 ligand, a CD40 ligand, erythropoeitin, thrombopoeitin, calcitonin, Fas ligand, ligand for receptor activator of NF-kappa B (RANKL), TNF-related apoptosis-inducing ligand (TRAIL), ORK/Tek, thymic stroma-derived lymphopoietin, granulocyte colony stimulating factor, granulocyte-macrophage colony stimulating factor, mast cell growth factor, stem cell growth factor, epidermal growth factor, RANTES, growth hormone, insulin, insulinotropin, insulin-like growth factors, parathyroid hormone, interferons (e.g., interferon beta), nerve growth factors, glucagon, interleukins 1 through 18, colony stimulating factors, lymphotoxin-β, tumor necrosis factor, leukemia inhibitory factor, oncostatin-M, various ligands for cell surface molecules Elk and Hek (such as the ligands for eph-related kinases, or LERKS), and antibody light or heavy chains.

Receptors (or soluble fragments thereof) for any of the aforementioned proteins can also be expressed using the inventive methods and compositions, including both forms of tumor necrosis factor receptor (referred to as p55 and p75), Interleukin-1 receptors (type 1 and 2), Interleukin-4 receptor, Interleukin-15 receptor, Interleukin-17 receptor, Interleukin-18 receptor, granulocyte-macrophage colony stimulating factor receptor, granulocyte colony stimulating factor receptor, receptors for oncostatin-M and leukemia inhibitory factor, receptor activator of NF-kappa B (RANK), receptors for TRAIL, BAFF receptor, lymphotoxin beta receptor, TGFβ receptor types I and II, and receptors that include death domains, such as Fas or Apoptosis-Inducing Receptor (AIR).

Other proteins that can be expressed using the recombination cassette and/or vectors of the invention include cluster of differentiation antigens (referred to as CD proteins): for example, those disclosed in Leukocyte Typing VI (Proceedings of the VIth International Workshop and Conference; Kishimoto, Kikutani et al., eds.; Kobe, Japan, 1996), or CD molecules disclosed in subsequent workshops. Examples of such molecules include CD27, CD30, CD39, CD40, and ligands thereto (CD27 ligand, CD30 ligand and CD40 ligand). Several of these are members of the TNF receptor family, which also includes 41BB and OX_{40;} the ligands are often members of the TNF family (as are 41BB ligand and Ox₄₀ ligand); accordingly, members of the TNF and TNFR families can also be expressed using the invention.

Polypeptides that are enzymatically active can also be expressed according to the invention. Examples include metalloproteinase-disintegrin family members, various kinases, glucocerebrosidase, superoxide dismutase, tissue plasminogen activator, Factor VII, Factor IX, apolipoprotein E, apolipoprotein A-I, globins, an IL-2 antagonist, alpha-1 antitrypsin, TNF-alpha Converting Enzyme (TACE), and numerous other enzymes. Ligands for enzymatically active proteins can also be expressed using the cassette and vector of the invention.

The inventive compositions and methods are also useful for expression of other types of recombinant proteins and polypeptides, including immunoglobuiin molecules or portions thereof and chimeric antibodies (e.g., an antibody having a human constant region coupled to a murine antigen binding region) or fragments thereof. Numerous techniques are known by which DNAs encoding immunoglobulin molecules can be manipulated to yield DNAs encoding recombinant proteins such as single chain antibodies, antibodies with enhanced affinity, or other antibody-based polypeptides (see, for example, Larrick et al., Biotechnology 7:934-938 (1989); Reichmann et al., Nature 332:323-327 (1988); Roberts et al., Nature 328:731-734 (1987); Verhoeyen et al., Science 239:1534-1536 (1988); Chaudhary et al., Nature 339:394-397 (1989)). Cloned humanized antibodies include those specifically binding to lymphotoxin beta-receptor and integrins such as VLA-1, VLA-4, and αvβ6, Such antibodies can be agonists or antagonists.

Various fusion proteins can also be expressed using the inventive methods and compositions. Examples of such fusion proteins include proteins expressed as a fusion with a portion of an immunoglobulin molecule, proteins expressed as fusion proteins with a zipper moiety, and novel polyfuictional proteins such as a fusion protein of a cytokine and a growth factor (e.g., GM-CSF and IL-3, MGF and IL-3). WO 93/08207 and WO 96/40918 describe the preparation of various soluble oligomeric forms of a molecule referred to as CD40L, including an ininunoglobulin fusion protein and a zipper fusion protein, respectively; the techniques discussed therein are applicable to other proteins.

Once a polynucleotide of interest is expressed, the expression product (e.g., a protein or polypeptide) may be purified using standard techniques in the art. For example, where the polynucleotide of interest encodes a fusion polypeptide comprising a purification tag, the polypeptide may be purified using antibodies that specifically bind to the tag. In one aspect an oligonucleotide encoding a tag molecule is ligated at the 5′ or 3′ end of a polynucleotide of interest encoding a desired polypeptide; the oligonucleotide may encode a polyHis (such as hexaHis), or other “tag” such as FLAG, HA (hemaglutinin Influenza virus) or myc for which commercially available antibodies exist. This tag is typically fused to the polypeptide upon expression of the polypeptide, and can serve as means for affinity purification of the desired polypeptide from the host cell. Affinity purification can be accomplished, for example, by column chromatography using antibodies against the tag as an affity matrix. Optionally, the tag can subsequently be removed from the purified polypeptide by various means such as proteolytic cleavage.

The recombination cassette and vectors of the invention can be used to provide a stable transfer of a polynucleotide of interest into a host cell. A stable transfer means that the polynucleotide of interest is continuously maintained in the host.

The vectors containing the polynucleotide of interest can be transferred into the host cell by well-known methods, depending on the type of cellular host. For example, micro-injection is commonly utilized for target cells, although calcium phosphate treatment, electroporation, lipofection, biolistics or viral-based transfection also may be used. Other methods used to transform mammalian cells include the use of Polybrene, protoplast fusion, and others (see, generally, Sambrook et al., Molecular Cloning: A Laboratory Manual, 2d ed., 1989, Cold Spring Harbor Laboratory Press, Cold Spring Harbor, N.Y, which is incorporated herein by reference).

In another aspect, the invention features a kit. The kit includes a recombination cassette and/or vector of the invention. The kit may further comprise a host cell comprising a target site (e.g., an FRT site). Such a host cell is typically provided in one or more sealed containers (e.g., packet, vial, tube, or microtiter plate), which in some embodiments also can contain nutritional media. A kit typically includes literature describing the properties of the host (e.g., its genotype) and/or instructions regarding its use for transformation. In some embodiments, a kit includes one or more polynucleotides comprising a recombination cassette and/or vector of the invention and may also include enzymes (e.g., a Flp recombinase) in a separate containers.

### EXAMPLES

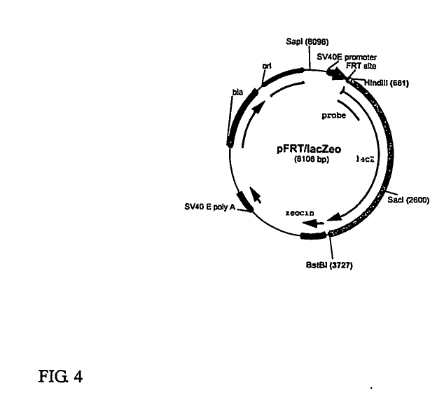

Generation of recombination cassette/vector. Plasmid pFRT/lacZeo (Invitrogen Inc., catalog #V6015-20; see FIG. 4) was linearized with SapI restriction endonuclease (1 unit SapI endonuclease per 1 μg plasmid) and was digested at 37° C. for approximately 16 hours. The host used for transfections was the dihydrofolate reductase (DIHR) deficient Chinese hamster ovary cell line DG44 (Urlaub et al., Cell 33, 405-412 (1983)). Approximately 2×10⁶ viable CHO-DG44 host cells were used per transfection run. The host cells were electroporated at 280V, 960 μF using 500 ng linearized pFRT/lacZeo plasmid per transfection.

Successful transfectants were selected in media containing 200 μg/ml Zeocin™ antibiotic (Invitrogen, Inc. cat. # R250-01). Colonies successfully growing in selective media were isolated by picking into 24-well cell culture plates. These isolates were then expanded in 6 well culture plates until they were sufficiently robust to transfer the transfected cells to T225 flasks. Genomic DNA was harvested from the transfected cells (5×10⁶ cells).

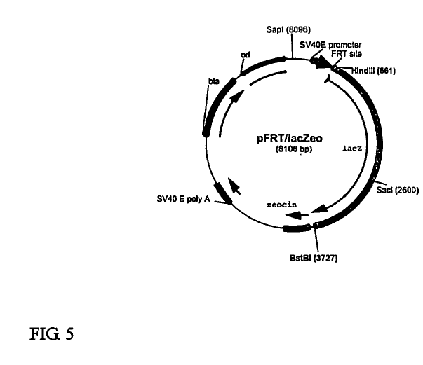

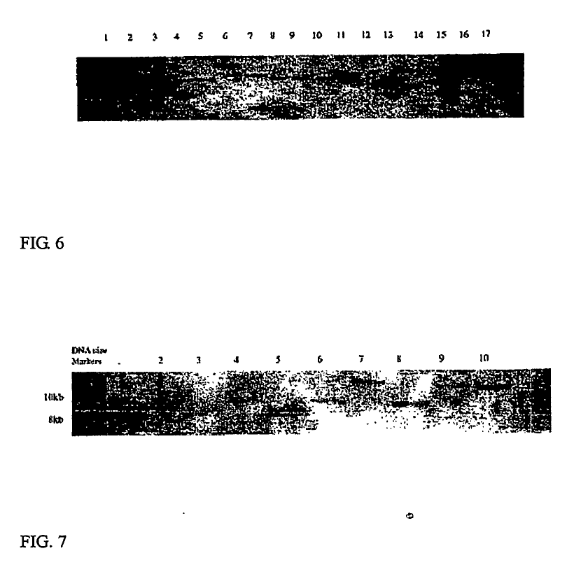

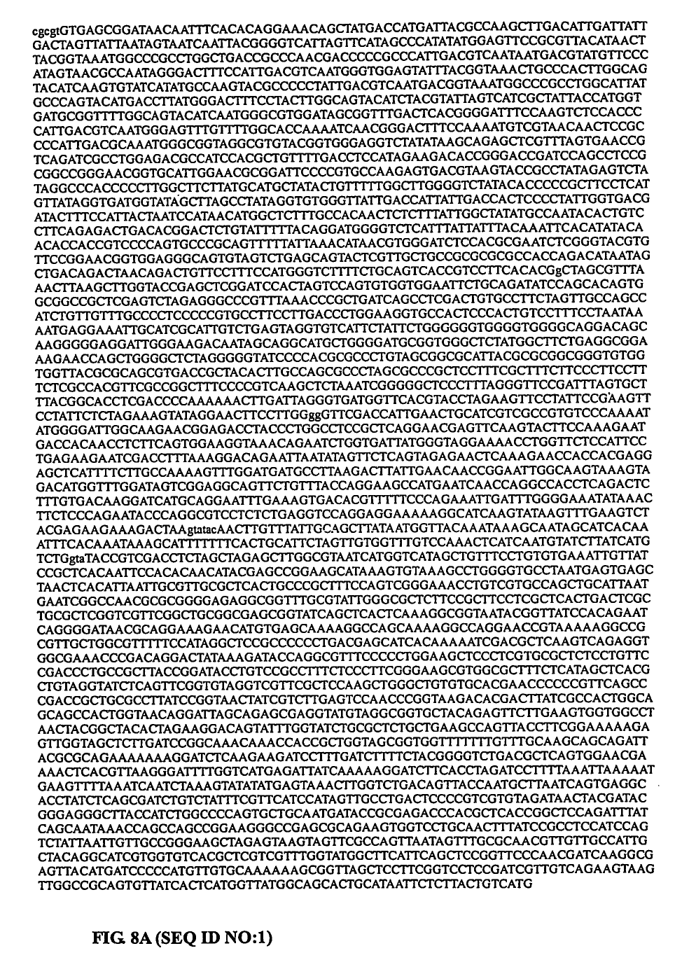

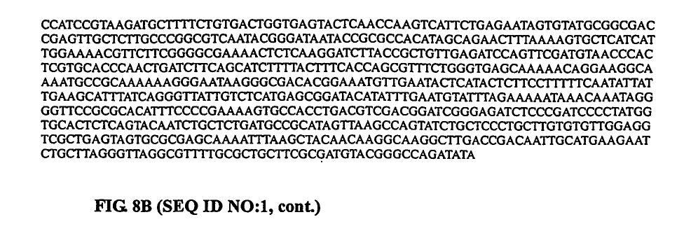

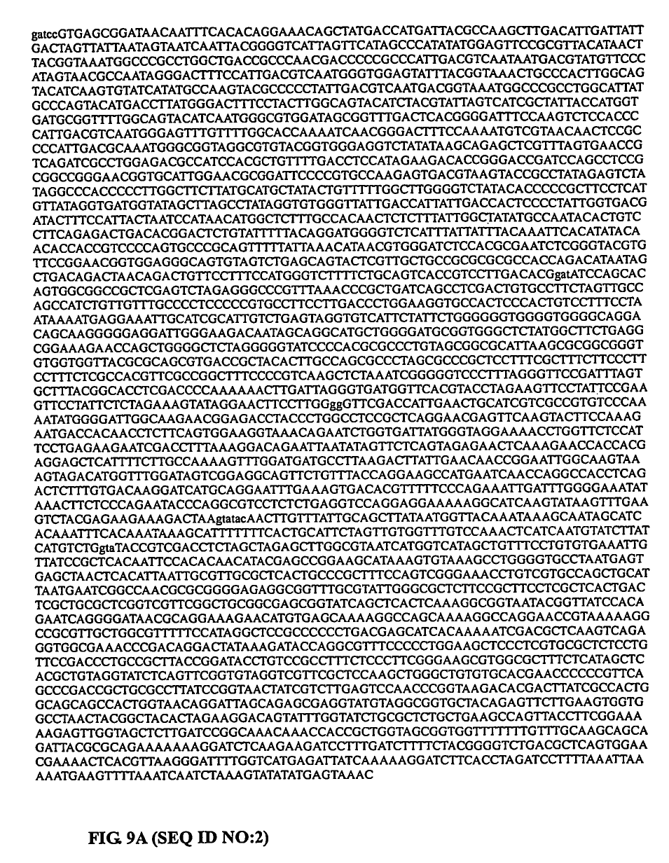

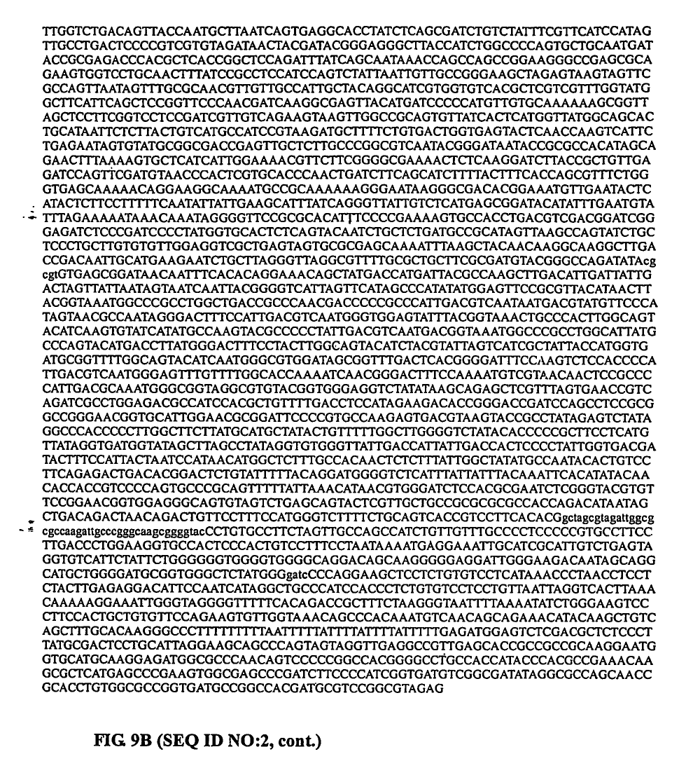

Genomic DNA of>100 cell lines was examined by Southern blot to verify the presence of a single copy of an FRT sequence. Sequence of the probe spans the FRT sequence and the 5′ end of the beta-galactosidase fusion gene (see FIG. 5). Of the >100 cell lines screened only 35 cell lines with a single FRT integration site were chosen for protein expression studies (See FIGS. 6 and 7).

Cloning of the reporter genes. The recombination cassette/vector including the CMV IE1, intron A fragment, and polyA signal domain were compared with a commercially available recombination vector.

Secreted Alkaline Phosphate (SEAP) was used as a model of secreted proteins to determine expression levels using a CHO-DG44 Flp-In host cell line. The SEAP coding sequence was obtained from the pSEAP2 expression plasmid (Clontech, Palo Alto, Calif.) using polymerase chain reaction (PCR) amplification. The 5′ primer was designed with a KpnI site, and the 3′ primer was designed with a BgIII site. 5′ priuner:

```
5′ primer:
(SEQ ID NO:4)
TTTTGGTACCATGCTGCTGCTGCTG (the start codon in bold)
          KpnI
3′ primer:
(SEQ ID NO.:5)
TTTTAGATCTCATGTCTGCTCGAAGCGGCC (termination codon in bold)
        BglII
```

The PCR was carried out as follows: 3 μg pSEAP2 plasmid (Clontech, Palo Alto, Calif.); 1 μM each 5′ and 3′ primers (see above); 0.25 mM dNTPs (Promega, Madison, Wis.); 2 units Vent® polymerase (New England Biolabs, Beverly, Mass.); 1× Vent® polymerase reaction buffer (New England Biolabs, Beverly, Mass.). The PCR was performed in a 100 μl reaction. PCR was carried out for 30 cycles of 95° C. for 30 seconds, 55° C. for 45 seconds, and 75° C for 2 minutes, followed by 75° C. for 10 minutes and held at 4° C. A PCR product of approximately 1.6 kb was obtained corresponding to the SEAP coding region. The PCR products were purified from the PCR mixture using the Wizard® PCR purification kit (Promega, Madison, Wis.) and eluted in dH₂O. The PCR product was digested first with endonuclease KpnI (New England Biolabs, Beverly, Mass.), followed by BgM (New England Biolabs, Beverly, Mass.).

Ten ng of the pFRT/dhfr-1 plasmid, which had been previously digested with KpnI and BamHI, was ligated with the digested SEAP PCR product. The SEAP coding region and junction points were sequenced. As expected, results matched the hypothetical sequence file. The plasmid was named pFRT/SEAP.

Transfection of the SEAP reporter gene into a Flp-In host cell line was accomplished by electroporation. Ten μg of pFRT/SEAP was combined with 90 μg of pOG44 (Invitrogen, Carlsbad, Calif.), the plasmid expressing the Flp recombinase. The DNA was ethanol precipitated, washed in 70% ethanol and dried. 2×10⁶ viable cells of the cell line were used for the transfection. The cells and DNA were combined aseptically into 800 μL sterile HeBS (20 mM Hepes pH=7.05, 137 mM NaCl, 5 mM KCl, 0.7 mM Na₂HPO₄, 6 mM dextrose) and transferred into a 0.4 cm cuvette (BioRad, Hercules, Calif.). Electroporation was done using the Gene Pulser® (Biorad, Hercules, Calif.) set at 0.28 kV and 950 μFd. After the electroporation pulse, the cells were allowed to incubate in the cuvette for 5-10 min at room temperature. They were then transferred to a centrifuge tube containing 10 ml of alpha-MEM plus nucleosides (Gibco, Gaithersburg, Md.) with 10% dialyzed fetal bovine serum (dFBS) (Hyclone, Logan, Utah) and pelleted at 1000 RPM for 5 min. Resuspended pellets were seeded into 6-well plates in alpha-MEM without nucleosides with 10% dFBS and incubated at 36° C. with 5% CO₂ in a humidified incubator for approximately 2 weeks, at which point colonies had formed.

Stable transfectants were analyzed as isolates. Isolates were obtained by “picking” colonies from the transfection. “Picking” was accomplished by aspirating directly over a colony with a P200 Pipetman™ set at 50 μl. The aspirated colony was transferred first to a 24 well plate. Once the well was >50% confluent, the media was exchanged to chemically-defined ProCHO4 media (Cambrex, Walkersville, Md.), supplemented with 4 mM L-glutamine (Cambrex, Walkersville, Md.), and the cells transfected into 6 well plates. Specific productivities were assessed in 6 well plates at or near confluence.

The specific productivity was assessed in the SEAP assay by exchanging the medium with fresh medium, then sampling the medium and counting the cells after 4 days. The product titer was normalized for the cell number at the end of 4 days, and the productivities were expressed as SEAP activity per cell.

Conditioned medium was analyzed using the Great EscAPe™ SEAP Reporter System 3 (Clontech, Palo Alto, Calif.). This assay uses a fluorescent substrate to detect SEAP activity in the conditioned medium. The kit was used in a 96 well format according to the manufacturer's instructions. All standards and samples were diluted in fresh medium rather than the dilution buffer provided. Instead of performing one reading after 60 minutes, a reading was taken at 10 minutes and at 40 minutes, and the data used to express SEAP activity as relative fluorescent units per minute (RFU/min). The emission filter used with the Cytofluor II™ plate reader (PerSeptive Biosystems, Framingham, Mass.) was 460 nm instead of the recommended 449 nm.

The RFU/min values were normalized to a standard curve based on a standard provided with the kit. Because the standard provided was not quantitated, all values are relative. These relative values were normalized to cell numbers and the incubation period to generate relative specific productivities (SEAP activity per cell per day).

SEAP Expression in A1 Flp-In CHO-DG44 Host Cells

| | day 0 | day 4 | | | Normalized SEAP |
|---|---|---|---|---|---|
| cell | initial density | final density | Integral Cell Area | | activity/cell/day |
| line | (cells/mL) | (cells/mL) | (cell * day)/mL | RFU/min | (*1E6) |
| 1 | 2.0E+05 | 6.2E+05 | 1.5E+06 | 0.75 | 0.5 |
| 2 | 2.0E+05 | 6.3E+05 | 1.5E+06 | 0.71 | 0.5 |
| 3 | 2.0E+05 | 5.3E+05 | 1.4E+06 | 0.75 | 0.6 |
| 4 | 2.0E+05 | 8.3E+05 | 1.8E+06 | 0.75 | 0.4 |
| 5 | 2.0E+05 | 7.5E+05 | 1.7E+06 | 0.71 | 0.4 |
| 6 | 2.0E+05 | 7.8E+05 | 1.7E+06 | 0.74 | 0.4 |
| 7 | 2.0E+05 | 1.5E+06 | 2.5E+06 | 0.74 | 0.3 |
| 8 | 2.0E+05 | 5.6E+05 | 1.4E+06 | 0.75 | 0.5 |
| 9 | 2.0E+05 | 4.8E+05 | 1.3E+06 | 0.72 | 0.6 |
| 10 | 2.0E+05 | 4.7E+05 | 1.3E+06 | 0.75 | 0.6 |
| 11 | 2.0E+05 | 5.3E+05 | 1.4E+06 | 0.74 | 0.5 |
| 12 | 2.0E+05 | 3.8E+05 | 1.1E+06 | 0.73 | 0.7 |
| 13 | 2.0E+05 | 7.6E+05 | 1.7E+06 | 0.74 | 0.4 |
| 14 | 2.0E+05 | 5.2E+05 | 1.3E+06 | 0.74 | 0.6 |
| 15 | 2.0E+05 | 5.4E+05 | 1.4E+06 | 0.72 | 0.5 |
| 16 | 2.0E+05 | 4.9E+05 | 1.3E+06 | 0.74 | 0.6 |
| 17 | 2.0E+05 | 5.0E+05 | 1.3E+06 | 0.73 | 0.6 |
| 18 | 2.0E+05 | 1.4E+06 | 2.4E+06 | 0.75 | 0.3 |
| 19 | 2.0E+05 | 5.2E+05 | 1.3E+06 | 0.76 | 0.6 |
| 20 | 2.0E+05 | 6.6E+05 | 1.5E+06 | 0.75 | 0.5 |

average RFU/min = 0.74 ± 0.01
average SEAP activity/cell/day * 1E6 = 0.5 ± 0.1

A number of embodiments of the invention have been described. Nevertheless, it will be understood that various modifications may be made without departing from the spirit and scope of the invention. Accordingly, other embodiments are within the scope of the following claims.

What is claimed is:

1. A recombination cassette comprising
a promoter/enhancer region;
a polynucleotide of interest;
a polyA signal domain; an FRT recombination domain; and
a dhfr polynucleotide,
wherein the promoter/enhancer region, the polynucleotide of interest and the polyA signal domain are operably linked.

2. The recombination cassette of claim 1, wherein the promoter/enhancer region comprises a human CMV immediate early 1 (hCMV IE1).

3. The recombination cassette of claim 2, wherein the hCMV IE₁ promoter/enhancer region comprises a sequence as set forth from about x₁ to about x₂ of SEQ ID NO:1 or 2, wherein x₁ is a nucleotide from position 1 to position 70 and x₂ is a nucleotide from position 770 to position 780.

4. The recombination cassette of claim 1, farther comprising a variable length intervening sequence (VLIVS) comprising a splice donor site and a splice acceptor site.

5. The recombination cassette of claim 4, wherein the VLIVS comprises an intron A of a hCMV IE1 gene.

6. The recombination cassette of claim 4, wherein the VLIVS comprises an intron A of a hCMV IE1 gene that has a deletion between the splice donor site and splice acceptor site of the intron A.

7. The recombination cassette of claim 6, wherein the VLIVS comprises a sequence from about x₃ to about x₄ of SEQ ID NO:1, wherein x₃ is a nucleotide from 770-780 and x₄ is a nucleotide from 1300-1310 of SEQ ID NO:1; or from about x₅ to about x₆ of SEQ ID NO:2, wherein x₅ is a nucleotide from 770-780 and x₆ is a nucleotide from 1300-1310 of SEQ ID NO:2.

8. The recombination cassette of claim 1, wherein the polynucleotide of interest encodes a therapeutic agent.

9. The recombination cassette of claim 1, wherein the polyA signal domain comprises at least 100 contiguous nucleotides of SEQ ID NO:3.

10. The recombination cassette of claim 9, wherein the polyA signal domain comprises SEQ ID NO:3.

11. A recombination vector comprising a recombination cassette of claim 1.

12. The recombination vector of claim 11, further comprising
a second promoter/enhancer region;
a second polynucleotide of interest; and
a second polyA signal domain,
wherein the second promoter/enhancer region, the second polynucleotide of interest, and the second polyA signal are operably linked.

13. The recombination vector of claim 12, further comprising an intervening domain between the second promoter/enhancer region and the second polynucleotide of interest.

14. A host cell comprising a recombination vector of claim 11.

15. The host cell of claim 14, wherein the host cell is adapted for growth in suspension.

16. The host cell of claim 14, wherein the host cell is adapted for growth in serum-free medium.

17. The host cell of claim 15, wherein the host cell is adapted for growth in serum-free medium.

18. A host cell comprising a recombination cassette of claim 1.

19. The host cell of claim 18, wherein the host cell is adapted for growth in suspension.

20. The host cell of claim 18, wherein the host cell is adapted for growth in serum-free medium.

21. The host cell of claim 19, wherein the host cell is adapted for growth in serum-free medium.

22. A recombination system comprising:
a recombination cassette of claim 1; and
a host cell comprising an FRT site.

23. The recombination system of claim 22, wherein the host cell is a CHO cell.

24. The recombination system of claim 23, wherein the CHO cell is a CHO-DG44 cell.

25. The recombination system of claim 22, wherein the host cell is adapted for growth in suspension.

26. The recombination system of claim 22, wherein the host cell is adapted for growth in serum-free medium.

27. The recombination system of claim 22, wherein the host cell is derived from a CHO-DG44 cell.

28. The recombination system of claim 22, wherein the host cell is dhfr⁻.

29. A kit comprising a vector of claim 10 and a host cell comprising an FRT site.

30. The kit of claim 29, wherein the host cell is a dhfr CHO host cell, the genome of which comprises an FRT site.


SEQUENCE LISTING (5 sequences)

SEQ ID NO: 1 - DNA, 5130 bp, Artificial Sequence
  Synthetic construct
cgcgtgtgagcggataacaatttcacacaggaaacagctatgaccatgattacgccaagcttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggaacggtggagggcagtgtagtctgagcagtactcgttgctgccgcgcgcgccaccagacataatagctgacagactaacagactgttcctttccatgggtcttttctgcagtcaccgtccttcacacggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattacgcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtacctagaagttcctattccgaagttcctattctctagaaagtataggaacttccttgggggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaagtatacaacttgtttattgcagcttataatggttacaaataaagcaatagcatcacaaatttcacaaataaagcatttttttcactgcattctagttgtggtttgtccaaactcatcaatgtatcttatcatgtctggtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatata

SEQ ID NO: 2 - DNA, 7245 bp, Artificial Sequence
  Synthetic construct
gatccgtgagcggataacaatttcacacaggaaacagctatgaccatgattacgccaagcttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggaacggtggagggcagtgtagtctgagcagtactcgttgctgccgcgcgcgccaccagacataatagctgacagactaacagactgttcctttccatgggtcttttctgcagtcaccgtccttgacacggatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggtccctttagggttccgatttagtgctttacggcacctcgaccccaaaaaacttgattagggtgatggttcacgtacctagaagttcctattccgaagttcctattctctagaaagtataggaacttccttgggggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaagtatacaacttgtttattgcagcttataatggttacaaataaagcaatagcatcacaaatttcacaaataaagcatttttttcactgcattctagttgtggtttgtccaaactcatcaatgtatcttatcatgtctggtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgtgagcggataacaatttcacacaggaaacagctatgaccatgattacgccaagcttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggaacggtggagggcagtgtagtctgagcagtactcgttgctgccgcgcgcgccaccagacataatagctgacagactaacagactgttcctttccatgggtcttttctgcagtcaccgtccttcacacggctagcgtagattggcgcgccaagattgcccgggcaagcggggtaccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatccccaggaagctcctctgtgtcctcataaaccctaacctcctctacttgagaggacattccaatcataggctgcccatccaccctctgtgtcctcctgttaattaggtcacttaaacaaaaaggaaattgggtaggggtttttcacagaccgctttctaagggtaattttaaaatatctgggaagtcccttccactgctgtgttccagaagtgttggtaaacagcccacaaatgtcaacagcagaaacatacaagctgtcagctttgcacaagggccctttttttttaatttttattttattttatttttgagatggagtctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagag

SEQ ID NO: 3 - DNA, 2660 bp, Homo sapiens
gaattcagcactgaatcatgcccagaacccccgcaatctattggctgtgctttggccccttttcccaacacacacattctgtctggtgggtggaggggaaacatgcggggaggaggaaaggaataggatagagagtgggatggggtcggtaggggtctcaaggactggcctatcctgacatccttctccgcgttcaggttggccaccatggcctgctgccagagggcacccacgtgacccttaaagagaggacaagttgggtggtatctctggctgacattctgtgcacaaccctcacaacgctggtgatggtgggaagggaaagatgacaagtcagggggcatgatcccagcatgtgtgggaggagcttctaaattatccattagcacaagcccgtcagtggccccaggcctaaacatgcagagaaacaggtgaggagaagcagcgagagagaaggggccaggtataaaaagggcccacaagagaccagctcaaggatcccaaggcccaactccccgaaccactcagggtcctgtggacagctcactagcggcaatggctgcaggtaagcgcccctaaaatccctttggcacaatgtgtcctgaggggagaggcggcgtcctgtagatgggacgggggcactaaccctcaggtttggggcttatgaatgttagctatcgccatctaagcccagtatttggccaatctctgaatgttcctggtccctggaggaggcagagagagagagagagaaaaaaaaaacccagctcctggaacagggagagcgctggcctcttgctctccagctccctctgttgcctccggtttctccccaggctcccggacgtccctgctcctggcttttggcctgctctgcctgtcctggcttcaagagggcagtgccttcccaaccattcccttatccaggctttttgacaacgctatgctccgcgcccgtcgcctgtaccagctggcatatgacacctatcaggagtttgtaagctcttgggtaatgggtgcgcttcagaggtggcaggaaggggtgaatttcccccgctgggaagtaatgggaggagactaaggagctcagggttgttttctgaagtgaaaatgcaggcagatgagcatacgctgagtgaggttcccagaaaagtaacaatgggagcaggtctccagcatagaccttggtgggcggtccttctcctaggaagaagcctatatcctgaaggagcagaagtattcattcctgcagaacccccagacctccctctgcttctcagagtctattccaacaccttccaacagggtgaaaacgcagcagaaatctgtgagtggatgccttctccccaggtgggatggggtagacctgtggtcagagcccccgggcagcacagccactgccggtccttcccctgcagaacctagagctgctccgcatctccctgctgctcatccagtcatggctggagcccgtgcagctcctcaggagcgtcttcgccaacagcctggtgtatggcgcctcggacagcaacgtctatcgccacctgaaggacctagaggaaggcatccaaacgctgatgtgggtgagggtggcaccaggatccaatcctggggccccactggcttccagggactggggagagaaacactgctgccctctttttagcagtcaggcgctgacccaagagaactcaccgtattcttcatttcccctcgtgaatcctccaggcctttctctacaacctggaggggagggaggaaaatggatgaatgagagagggagggaacagtgcccaagcgcttggcctctccttctcttccttcactttgcagaggctggaagatggcagcccccggactgggcagatcttcaatcagtcctacagcaagtttgacacaaaatcgcacaacgatgacgcactgctcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggccaggttgggaagacaacctgtagggccttcagggtctattcgggaaccaggctggagtgcagtggcagtcttggctcgctgcaatctccgcctcctgggttcaagcgattctcctgcctcagtctcccgaatagttgcgattccaggcatgcaagaccaggctcagctaatttttgtatttttggtagagacggggtttcaccatattggccagtctggtctccatctcctgacctcaggtaatccgcccgcctcggcctcccaaattgctgggattacaggtatgagccactgggcccttccctgtcctgtgattttaaaataattataccagcagaaggacgtccagacacagcatgggctacctggccatgcccagccagttggacatttgagttgtttgcttggcactgtcctctcatgcattgggtccactcagtagatgcttgttgaattc

SEQ ID NO: 4 - DNA, 25 bp, Artificial Sequence
  Primer
ttttggtaccatgctgctgctgctg

SEQ ID NO: 5 - DNA, 30 bp, Artificial Sequence
  Primer
ccggcgaagctcgtctgtactctagatttt